(19) United States
(12) Patent Application Publication
(10) Pub. No.: US 2026/0298824 A1
(43) Pub. Date: Oct. 1, 2026

(54) NANOWIRE ARRAY CONTAINING NONSTICK LAYER AND METHODS OF MAKING AND USING THEROF FOR ANALYTE DETECTION

(71) Applicant: ALIGNEDBIO AB, Lund (SE)

(72) Inventors: Niklas MÅRTENSSON, Malmö (SE); Søren ECHWALD, Humlebaek (DE); YuYu LI, Lund (SE); Patrick FLATT, Malmö (SE)

(21) Appl. No.: 19/479,901

(22) PCT Filed: May 16, 2024
(86) PCT No.: PCT/IB2024/054776

§ 371 Date: Oct. 29, 2025

Related U.S. Application Data
(60) Provisional application No. 63/503,029, filed on May 18, 2023.

(51) Int. Cl. G01N 21/64; G01N 33/543; G01N 33/58

(52) U.S. Cl. CPC G01N 21/6428; G01N 33/54366; G01N 33/582; G01N 2021/6439

(57) ABSTRACT

A nanowire array includes nanowires aligned with one another, and configured to bind with a target, and a nonstick layer surrounding fixed ends of the nanowires, the nonstick layer having a lower binding affinity with the target than the nanowires.

### FIELD

The present disclosure relates to nanowire devices and methods, and in particular, to nanowire arrays including a nonstick layer, and methods of making and using thereof.

### BACKGROUND

In standard analyte (e.g., biomolecule) detection methods, primary recognition molecules (i.e., so called capture molecules, which are also known as binding molecules), such as antibodies or oligonucleotides, are immobilized on flat solid supports, e.g. in microwell plates. Analyte (e.g., biomolecule) capture by the immobilized primary recognition molecules is the basis for detecting the presence of the analyte. This capture may be detected by specific binding of another set of secondary recognition molecules which recognize another region of the analyte than the one mediating binding to the flat solid support. Often these secondary recognition molecules comprise or are labeled with fluorophores. These methods utilize fluorescence linked immunosorbent assays (FLISAs) when fluorescence-based detection is used. The secondary recognition molecules may be bound to the analyte before or after the analyte binds to the primary analyte molecule(s).

### SUMMARY

Various embodiments provide a nanowire array includes nanowires aligned with one another, and configured to bind with a target, and a nonstick layer surrounding fixed ends of the nanowires, the nonstick layer having a lower binding affinity with the target than the nanowires.

Various embodiments provide a sensing method comprises providing a nanowire array nanowires aligned with one another, capture molecules bound to the nanowires, and a nonstick layer surrounding fixed ends of the nanowires, the nonstick layer having a lower binding affinity with the capture molecules than the nanowires, providing an analyte fluid containing an analyte labeled with a fluorophore to the nanowire array, selectively binding the analyte labeled with the fluorophore to the capture molecules, exposing the array to source radiation, waveguiding emitted radiation from the fluorophores through the nanowires to a radiation detector, and determining a presence of the analyte in the analyte fluid based on detected emitted radiation.

Various embodiments provide a method of forming a nanowire array comprises providing a nanowire array comprising nanowires aligned with one another and a nonstick layer surrounding fixed ends of the nanowires, and providing target molecules to the nanowire array such that the target molecules selectively bind to the nanowires relative to the nonstick layer.

### BRIEF DESCRIPTION OF THE DRAWINGS

FIG. 1 is a schematic of an epitaxial nanowire growth process for forming a nanowire array, according to various embodiments of the present disclosure.

FIG. 2 is a schematic of an Aerotaxy™ nanowire growth process for forming a nanowire array, according to various embodiments of the present disclosure.

FIG. 3 is a schematic illustration of an aggregate of aligned and assembled nanowires at a fluid interface, according to various embodiments of the present disclosure.

FIG. 4 is a schematic view of a nanowire transfer apparatus 60 for assembling the nanowires and forming a nanowire array, according to various embodiments of the present disclosure.

FIG. 5 is a cross-sectional view of a nanowire array after exposure to a target, according to various embodiments of the present disclosure.

FIG. 6 is a cross-sectional view of a comparative nanowire array that does not include a nonstick layer after exposure to a target.

FIG. 7 is a perspective view of a method of using the nanowire array according to various embodiments of the present disclosure.

### DETAILED DESCRIPTION

Various embodiments will be described below with reference to the drawings. The embodiments are to be seen as exemplary, and other ways of realizing the solutions provided within the scope of the claims are therefore foreseeable.

In one embodiment, an analyte detection system includes nanowires positioned on the substrate, the nanowire configured to bind with a target (e.g., an analyte molecule or a capture molecule which is configured to bind to the analyte molecule) and a fluorophore, an excitation radiation source configured to irradiate the nanowire with excitation radiation, and a radiation detection device configured to collect radiation generated by the fluorophore and output from the free end of the nanowire. The nanowires increase the surface area available for target binding compared to a flat surface and function as waveguides for radiation emitted by the fluorophores.

However, some of the target molecules bind to the flat substrate surface exposed between the nanowires. The nanowires either do not function as waveguides or their waveguide function is decreased for radiation emitted from the fluorophores bound to the analyte molecules which are bound to the substrate rather than to the nanowires.

In one embodiment, a nonstick layer disposed on the flat substrate surface between the nanowires. The nonstick layer surrounds the fixed ends of the nanowires that face the substrate. The nonstick layer has a lower binding affinity with the target than the nanowires. The nonstick layer decreases the amount of analyte molecules and fluorophores that are bound to the flat nonstick layer surface exposed between the nanowires. This increases the proportion of the analyte molecules and fluorophores which are directly or indirectly bound to the nanowires, while increases the proportion of the radiation from the fluorophores which is waveguided by the nanowires toward the radiation detection device.

A nanowire is a structure with a diameter (or width in the case of hexagonal horizontal cross-sectional shaped nanowires, such as Group III-nitride based semiconductor nanowires) less than 1 micron, such as 10-500 nm, for example 20 to 200 nm. The length may be greater than 1 micron. The length to diameter/width ratio may be 10:1 or greater, such as 5:1 or greater. The nanowires may comprise any suitable material, such as semiconductor, insulator, conductor (e.g., metal). In one embodiment, the nanowires may comprise semiconductor nanowires (e.g., Group III-V semiconductor nanowires) which function as radiation waveguides. Semiconductor nanowires may be made of Group III-V, II-VI or IV semiconductor materials, such as GaAs, InP, InAs, GaAsₓP₁₋ₓ, InₓGa₁₋ₓP, InGaAsP, GaN, InN, GaₓIn₁₋ₓN, GaP, InSb, GaSb, InxAl₁₋ₓSb GaₓAl₁₋ₓSb, AlN, BN, Si or SiC. The semiconductor nanowires may have insulating shells on their sidewalls (e.g., core-shell type nanowires). The insulating shells may comprise silicon oxide (e.g., silicon dioxide), silicon nitride, silicon oxynitride, aluminum oxide or other inorganic oxide, carbide or nitride materials.

In one embodiment a nanowire array may be formed by an epitaxial growth process on the substrate. In another embodiment, the nanowires may be formed using a vapor phase process (e.g., an Aerotaxy™ process), followed by a nanowire alignment and capture process followed by attachment of the aligned, captured nanowires on a solid substrate. Devices made by the methods include, but are not limited to, nanowire arrays designed for the optical detection of target molecules (e.g., analyte biomolecules) and lab-on-chip devices.

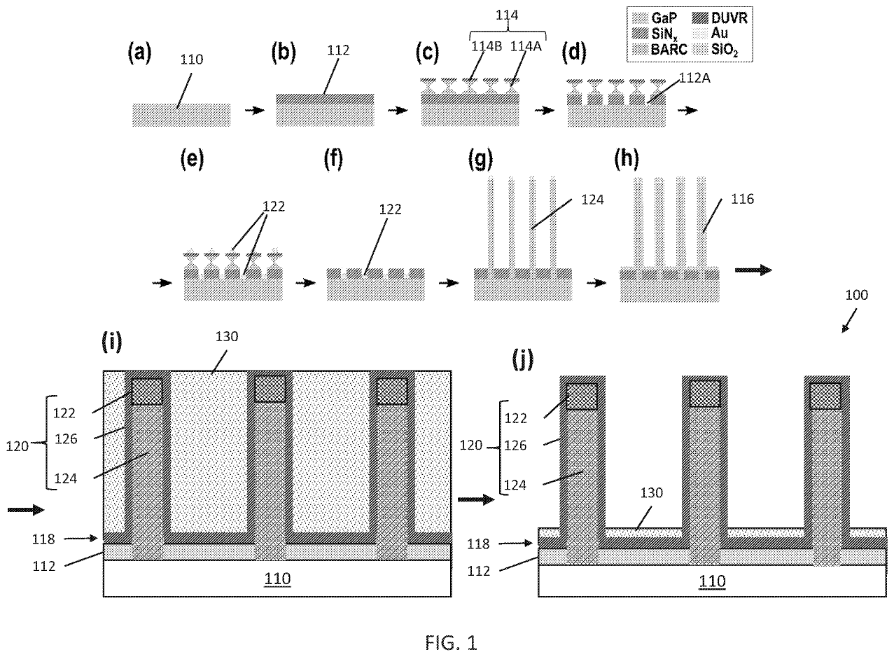

FIG. 1 is a schematic steps of an epitaxial nanowire growth process on a substrate for forming a nanowire array 100, according to one embodiment of the present disclosure. Referring to FIG. 1, in step (a) a substrate 102, such as a GaP(111)B wafer substrate is provided. Other substrate materials (e.g., other semiconductor materials, such as silicon, GaAs, GaN, etc., may be used for the substrate. In step (b), a growth mask layer 112 may be formed by any suitable method, such as by plasma-enhanced chemical vapor deposition (PECVD). The growth mask layer 112 may have a thickness ranging from about 60 nm to about 80 nm, such as about 70 nm, and may be formed of a dielectric material, such as silicon nitride or the like.

In step (c), the growth mask layer 112 may then be covered by an etch mask 114, such as double layer including a bottom anti-reflection coating (BARC) 114A and a photoresist 114B, such as a deep-ultraviolet resist (DUVR). BARC 114A may comprise an organic or an inorganic anti-reflective material. Other etch masks, such as other hard masks, photoresists or electron beam resists may be used. The photoresist 114B may be photolithographically patterned to expose portions of BARC 114A by displacement talbot lithography (DTL) or the like. The BARC layer 114A is then etched or developed (depending on its composition) to expose portions of the growth mask layer 112.

In step (d), an etching process, such as reactive ion etching (RIE), may be performed, using the patterned etch mask 114 as a mask to pattern the growth mask layer 112 to form openings 112A therein. In step (e), seed particles 122 are deposited on the patterned etch mask layer 114 and on the exposed surfaces of the substrate 110 in the openings 112A by any suitable method, such an evaporation method. The seed particles may comprise gold or gold alloy particles and have size ranging from about 50 nm to about 70 nm, such as about 60 nm. In step (f), a lift-off process may be performed to remove the etch mask 114, including the remaining seed particles 122 disposed thereon, such that the seed particles 122 remain on the substrate 110 within the openings 112A of the mask layer 112.

In step (g), semiconductor nanowires 124 are grown below the seed particles 122, by any suitable method. For example, the semiconductor nanowires 124 may comprise GaP nanowires formed by metal-organic vapor phase epitaxy (MOVPE) in a vapor-liquid-solid (VLS) mode, using an appropriate reactor (e.g., Aixtron 200/4). In particular, TMGa, phosphine, and hydrogen chloride (HCl) may be introduced into the reactor at about 480° C. TMGa and phosphine break down and their atoms (e.g., primarily gallium) are dissolved in the Au particles, forming a liquid (e.g., gold-gallium) alloy (which may optionally contain a small amount of phosphorus), which leads to supersaturation, and the nucleation of the GaP crystal phase. Other semiconductor material nanowires may alternatively be formed. If desired, the seed particles 122 may be removed selective etching after semiconductor nanowire growth.

In step (h), a dielectric material 116, such as silicon oxide (e.g., SiO₂) or the like, may be deposited over the substrate 110, at a thickness ranging from about 5 nm to about 15 nm, such as about 10 nm. The dielectric material 116 may be deposited by any suitable method, such as by atomic layer deposition (ALD), a wet chemical method or the like.

As shown in step (i), a vertical portion of the dielectric material 116 formed on the nanowires 124 forms nanowire shells 126 (i.e., dielectric shells around semiconductor nanowires 124). A horizontal portion of the dielectric material 116 forms a base layer 118 disposed on the growth mask layer 112. Accordingly, nanowires 120 may formed on the substrate 110. The nanowires 120 may each include an optional seed particle 122 (if not removed after growth), a semiconductor nanowire 124, and an optional nanowire shell 126. The nanowires 120 may extend in a direction that is substantially perpendicular to a plane (i.e., top surface) of the substrate 110. For example, an angle formed between the nanowires 120 and the plane of the substrate 112 may range from about 75 degrees to about 90 degrees, such as from about 80 degrees to 90 degrees. Each nanowire 120 may have a free end (e.g., an end including the seed particle 122, if present) and an opposing fixed end that faces the substrate 110 and in this embodiment that is attached to the substrate 110.

In step (i), a nonstick layer 130 may be formed on the base layer 118 and may surround the fixed ends of the nanowires 120. The nonstick layer 130 may include a polymeric material and may be formed by applying a monomer solution to the substrate 110, and then polymerizing the monomer to form the nonstick layer 130.

In optional step (j), if the thickness of the nonstick layer 130 is greater than desired (e.g., if it covers more than half of the height of the nanowires 120), then a selective etch-back process may be used to selectively reduce the thickness of the nonstick layer 130 and form the nanowire array 100. The selective etch-back may selectively etch the polymer nonstick layer 130 compared to the inorganic material nanowires 120. For example, the final nonstick layer 130 may have a thickness ranging from about 10 nm to about 500 nm, such as from about 20 nm to about 100 nm, or about 50 nm.

The nonstick layer 130 may have a lower binding affinity for a target material (e.g., analyte molecules, such as analyte biomolecules or primary recognition (i.e., binding or capture) molecules, which are configured to bind to the analyte molecules)) than the inorganic material of the nanowires 120, such as the dielectric material of the nanowire shells 126. In some embodiments, the nonstick layer 130 may be formed of a hydrophilic material, such as a hydrophilic polymer. For example, the nonstick layer 130 may be formed of any suitable hydrophobic or hydrophilic polymer, such as polyacrylate polymers (e.g., poly(propylene glycol) diacrylate (PPGDA)), polyethylene glycol (PEG) polymers, poly(ethylene oxide) (PEO) polymers, polyvinyl alcohol (PVA) polymers, poly(vinyl pyrrolidone) (PVP) polymers, copolymers thereof, combinations thereof, or the like. In some embodiments, the nonstick layer may be formed by polymerizing tri (propylene glycol) diacrylate monomers having the following Formula 1:

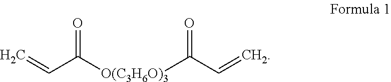

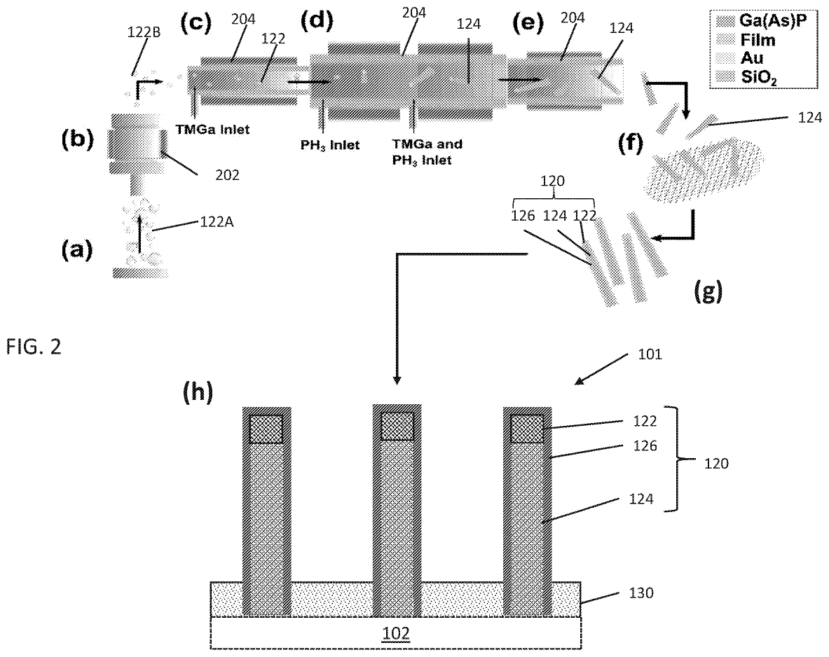

FIG. 2 is a schematic of a vapor phase (e.g., Aerotaxy™) nanowire growth process for forming a nanowire array 101, according another embodiment of the present disclosure. Referring to FIG. 2, a reactor may be used for the growth of gallium phosphide (GaP) or gallium arsenide phosphide GaAsP semiconductor nanowires 124, catalyzed by Au seed particles 122 in the absence of a substrate.

In particular, in step (a) growth catalyst (e.g., seed) particle agglomerates, such as gold agglomerates 122A may formed by evaporation-condensation in a high-temperature furnace or by another suitable method (e.g., electron beam evaporation). In step (b), the Au agglomerates are size-sorted (50±15 nm), in a differential mobility analyzer (DMA) 202, for a final selection of spherical seed particles (e.g., gold catalyst nanoparticles) 122B.

In step (c), the Au seed particles 122B may be provided to a growth reactor 204. The gold seed particles 122B are exposed to trimethylgallium (Ga(CH₃)₃) (TMGa), at a suitable growth temperature, such as 430° C. and under suitable pressure, such as atmospheric pressure, to initiate the formation of Ga—Au alloy seed particles 122. In step (d), the Au—Ga alloy seed particles 122 are carried into a phosphine (PH₃) atmosphere and reacted at a suitable temperature, such as 550° C., in the reactor 204. As P atoms dissolve, supersaturation and nucleation events occur, which are promoted by the continuous flow of precursors. This process favors one-dimensional semiconductor nanowire growth, guided by the seed particles 122 and the formation of crystalline semiconductor nanowires 124, such as GaP nanowires.

In step (e), the semiconductor wires 124 are cooled and carried out of the reactor 204 as an aerosol. In step (f), the semiconductor wires 124 are filtered and collected. In step (g), the semiconductor nanowires 124 are coated with a dielectric material, such as SiO₂ to form nanowire shells 126 on the semiconductor nanowires 124, and thereby form nanowires 120. The nanowires 120 comprise a seed particle 122, a semiconductor nanowire 124, and a nanowire shell 126. The nanowire shells 126 may have a thickness ranging from about 10 nm to 40 nm, such as about 30 nm.

In step (h), the nanowires 120 may be aligned with one another and embedded into a nonstick layer 130, to form a nanowire array 101. The nonstick layer 130 may be disposed on an optional substrate 102. The substrate 102 may comprise a semiconductor substrate (e.g., a silicon wafer), an insulating substrate (e.g., a plastic, glass or ceramic substrate) or a conductive substrate (e.g., a metal substrate). Alternatively, the substrate 102 may be omitted, and the non-stick layer 130 may comprise a free-standing film. Thus, the substrate 102 is shown in dashed lines in step (h).

The nonstick layer 130 may be a film or layer formed of a hydrophilic polymer material, as discussed above with respect to FIG. 1. For example, suitable hydrophobic polymers may include polyacrylate polymers, polyethylene glycol (PEG) polymers, poly(ethylene oxide) (PEO) polymers, polyvinyl alcohol (PVA) polymers, poly(vinyl pyrrolidone) (PVP) polymers, copolymers thereof, or the like.

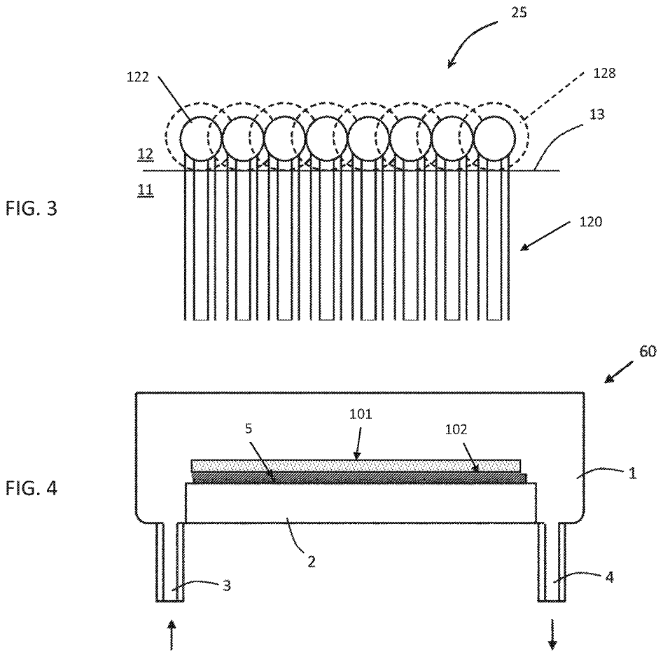

The nanowires 120 may be aligned using any suitable method, such as a method described below with respect to FIGS. 3-4. The nonstick layer 130 formed on the substrate 102 may have a thickness ranging from about 10 nm to about 500 nm, such as from about 20 nm to about 100 nm, or about 50 nm. Alternatively, if the nonstick layer 130 comprises a free-standing film, then it may have a thickness of 40 to 400 microns, such as 45 to 90 microns.

FIG. 3 is a schematic illustration of an aggregate 25 of aligned and assembled nanowires 120 at a fluid interface 13, according to various embodiments of the present disclosure. FIG. 4 is a schematic view of a nanowire transfer apparatus 60 for assembling the nanowires 120 and forming a nanowire array 101 from the fluid interface 13, according to various embodiments of the present disclosure.

Referring to FIGS. 3-4, the nanowires 120 formed at step (g) of FIG. 2 are collected and provided to the fluid interface 13 to provide the aggregate 25. The aggregate 25 may be disposed at the interface 13 between a first fluid 11 or phase and a second fluid 12 or phase, which may be disposed in the container 1 shown in FIG. 4. In some embodiments, the nanowires 120 may include a first functionalizing compound 128 that is attached to one end of the nanowires 120, such as to the seed particles 122. A second, different functionalizing compound (not shown) may be attached to an opposing second end of the nanowires 120 (e.g., to the dielectric shell 126). The functionalizing compound(s) 128 aid in aligning the nanowires 120 at the interface 13 between the first and second fluids 11, 12. In some embodiments, the functionalizing compound 128 may comprise 1-octadecanethiol (ODT) attached to the seed particles 122, while (12-phosphonododecyl)phosphonic acid (PPA)) may be attached to the dielectric shell 126. Further, the functionalizing compound 128 may have a functional group, or a different one at each ends of the functionalizing compounds 128. Various types of functionalizing components and examples of such types are given in Table I of PCT published application number WO2015/166416, which is hereby incorporated by reference, in its entirety.

The first and second fluids 11, 12 may be selected such that the fluids form distinct phases. For example, the first fluid 11 may be denser than the second fluid 12. Based on the choice of functionalizing compound(s) 128 and the type and composition of the fluids 11, 12, the orientation and alignment of the nanowires 120 may be controlled to form the aggregate 25 at the interface 13. In a similar manner, these parameters/compositional variables may allow the nanowire-nanowire interspacing to be varied, resulting in assemblies with different (i.e. pre-selected) densities (e.g. density of nanowires per square micron). The nanowire-nanowire interspacing can be deduced from the percentage of surface area covered after the capture of the aligned nanowires on the substrate 102.

The first fluid 11 may have a composition comprising a concentration of first substance of at least one of acetone, acetonitrile, dimethyl sulfoxide, di ethylene glycol, or isopropyl alcohol. This first substance is provided to at a certain concentration in water, i.e. in an aqueous solution. By including this first substance to a certain concentration in the first fluid 11, the interfacial energy between the first fluid 11 and the second fluid 12, which may be a liquid, is lowered and the nanowire surface charge density may be decreased. This contributes to the nanowires 120 predisposition to align vertically in the interface 13, and thus, a tighter packing in the aggregate 25.

Once a suitable aggregate 25 of nanowires 120 has formed at the interface 13, transfer of the aggregate to a substrate may be carried out in the apparatus 60. The apparatus 60 may include a vessel or container 1, and be made of any material suitable for holding the first and second fluids 11, 12. The container 1 may thus have inner walls of glass, metal or any other solid material. A support member 2 may be provided at an inner bottom part of the container 1, having a substantially horizontal support surface 5 for supporting a substrate 102. In one embodiment, the apparatus 60 may comprise ports or conduit 3, 4, for injecting and/or extracting fluids with respect to the container 1.

Fluid may be added or extracted from the container 1, such that the aggregate 25 of aligned nanowires 120 is brought into contact the substrate 102. In some embodiments, the first fluid 11 may include one or more polymer precursor(s). The polymer precursor may be polymerized to form the nonstick layer 130 (see step (h) in FIG. 2), in order to fix the nanowires in position on the substrate 102 and form the nanowire array 101. For example, the polymer precursors may be irradiated (e.g., exposed to UV radiation) or heated to initiate polymerization and/or crosslinking. In the alternative, the nonstick layer 130 may be formed by a solidification process. For example, the nonstick layer 130 may be formed by removing a solvent (e.g., water or organic solvent) from the first fluid 11 without chemical crosslinking, to leave the nanowires in the polymer precursor(s) on the substrate 102, followed by polymerization and/or crosslinking of the remaining polymer precursor(s) to form the nonstick layer 130 surrounding at least the bottom portions of the nanowires 120, as shown in step (h) of FIG. 2. A further description of nanowire alignment and transfer may be found in U.S. Pat. Nos. 10,692,719, and 10,919,074, which are hereby incorporated by reference, in their entireties.

The nanowire array 101 may be removed from the apparatus 60. In some embodiments, an etch-back process may optionally be performed to selectively reduce the thickness of the nonstick layer 130, as discussed above with regard to FIG. 1.

In an alternative embodiment, rather than aligning the nanowires 120 at an interface 13 between two liquids 11 and 12, the nanowires 120 may be aligned in the liquid 11. The liquid 11 may contain monomers of the nonstick layer 130. In this embodiment, the monomers in the liquid 11 are polymerized after the nanowires 120 are aligned. This results in a free-standing nonstick layer 130 surrounding at least the fixed ends (e.g., bottom ends) of the aligned nanowires 120. The free-standing nonstick layer 130 may be etched back to expose free ends of the aligned nanowires 120 to form the array 101 shown in step (h) of FIG. 2. In this embodiment, the substrate 102 may be omitted.

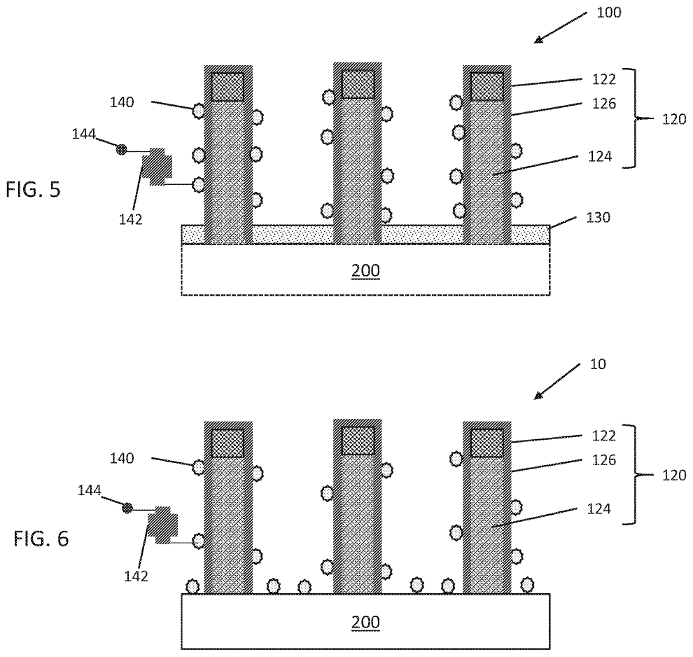

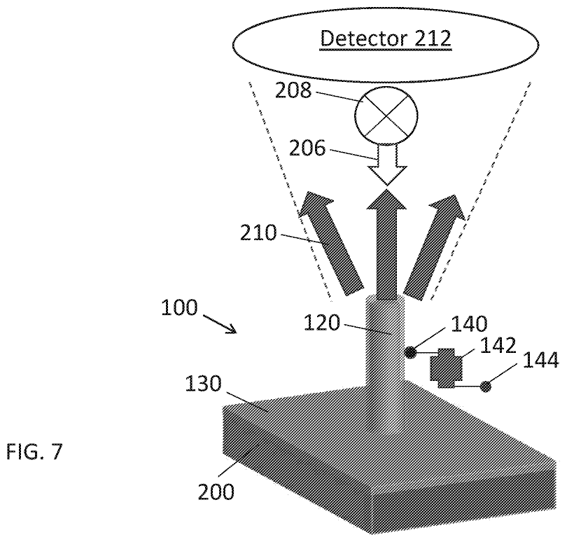

FIG. 5 is a cross-sectional view of a nanowire array 100 that includes the nonstick layer 130 after exposure to a target 140, according to various embodiments. FIG. 6 is a cross-sectional view of a comparative nanowire array 10 that does not include a nonstick layer 130 after exposure to a target 140. FIG. 7 is a perspective view of a method of using the nanowire array 100 according to various embodiments of the present disclosure. Only one nanowire 120 is shown in FIG. 7 for clarity.

The arrays 100, 10 may include an optional substrate 200, which may comprise the substrate 110 shown in FIG. 1 or the substrate 102 shown in FIG. 2 or another support which supports the nonstick layer 130. Alternatively, the substrate 200 may be omitted if the nonstick layer 130 comprises a free-standing film. In one embodiment, the target 140 may comprise primary recognition (i.e., binding or capture) molecules, such as biotin-conjugated bovine serum albumin (b-BSA) or the like. The primary recognition molecules may be used to selectively bind analyte molecules 142, such as biomolecules for example proteins, DNA or RNA, which are labeled with (e.g., bound to) fluorophores 144. For example, the analyte molecules 142 may comprise streptavidin (StvA647) which binds with high specificity to biotin (II), labeled with Alexa Fluor 647 fluorophores 144.

Referring to FIGS. 5 and 6, the target 140 may selectively bind to the hydrophobic shells 126 of the nanowires 120 in both nanowire arrays 100, 10. However, the nonstick layer 130 of the nanowire array 100 prevents or reduces binding between the target 140 and the substrate 200 (e.g., to the base layer 118 if present on the substrate 200 or to the top surface of the substrate if the base layer 118 is not present) in the nanowire array 100. In contrast, the target 140 has a higher binding affinity to the substrate 200 in the comparative nanowire array 10 which lacks the nonstick layer 130. If the substrate 200 in array 100 is omitted, then the nonstick layer 130 causes a higher fraction of the target 140 (e.g., greater than half, such as 60 to 99%) to bind to the nanowires 120 rather than to the nonstick layer 130.

The nanowire array 100 may be located in a microfluidic biosensor device or another biosensor device which permits an analyte fluid to flow past the nanowire array 100. After an analyte fluid (e.g., water or another solvent) containing the analyte molecules 142 labeled with fluorophores 144 is flown past the nanowires 120, the analyte molecules selectively bind to the target molecules 140 (e.g., the primary recognition (i.e., binding or capture) molecules). Since there is a larger proportion of the target molecules 140 bound to the nanowires 120 than to the flat surface of the substrate 200 or the nonstick layer 130 in array 100 than in array 10, a larger proportion of the analyte molecules 142 labeled with fluorophores 144 is bound to the nanowires 120 in array 100 than in array 10.

As shown in FIG. 7, the array 100 is exposed to source radiation 206 from a radiation source (e.g., lamp or laser) 208 which is in optical communication with the free (e.g., top) ends of the nanowires 120 which face away from the nonstick film 130. If the fluorophore 144 labeled analyte molecules 142 are bound to the target molecules 140 on the nanowires 120, then the fluorophores 144 emit radiation (e.g., visible light, UV or IR radiation) 210. The nanowires 120 waveguide the emitted radiation 210 from their free (e.g., top) end to a radiation detector 212 which is in optical communication with the free ends of the nanowires 120. If the radiation detector 212 detects the emitted radiation 210, then a controller correlates this to the presence and/or the concentration of the analyte molecules 142 in the analyte fluid. If no emitted radiation 210 is detected, then the analyte molecules 142 are not present in the analyte fluid. It should be noted that the radiation source 206 and the radiation detector 212 are in optical communication with the free ends of the nanowires 120 by either directly facing the free ends of the nanowires 120 or indirectly via mirrors or other beam guiding optics which guide the source radiation 206 and/or the emitted radiation 210 to and from the free ends of the nanowires 120.

Accordingly, the nonstick layer 130 beneficially prevents or reduces non-specific binding of target molecules 140 to areas of the nanowire array 100 other than the nanowires 120. Accordingly, the nonstick layer 130 increases the sensitivity of the nanowire array 100, by reducing competitive binding. In particular, detection efficiency may be increased by localizing the target 140 on the nanowires 120 which leads a higher proportion of the radiation from the fluorophores 144 to be waveguided by the nanowires 120 to the radiation detector. In addition, the nonstick layer 130 also strengthens the connection between the nanowires 120 and the substrate 200.

Although the foregoing refers to particular preferred embodiments, it will be understood that the invention is not so limited. It will occur to those of ordinary skill in the art that various modifications may be made to the disclosed embodiments and that such modifications are intended to be within the scope of the claims. All of the publications, patent applications and patents cited herein are incorporated herein by reference in their entirety.

What is claimed is:

1. A nanowire array, comprising:
nanowires aligned with one another, and configured to bind with a target; and
a nonstick layer surrounding fixed ends of the nanowires, the nonstick layer having a lower binding affinity with the target than the nanowires.

2. The nanowire array of claim 1, wherein:
each nanowire comprises a semiconductor nanowire surrounded by an inorganic dielectric shell; and
the nonstick layer comprises a polymer layer.

3. The nanowire array of claim 2, wherein the nonstick layer comprises a polyacrylate polymer, a polyethylene glycol (PEG) polymer, a poly(ethylene oxide) (PEO) polymer, a polyvinyl alcohol (PVA) polymer, a poly(vinyl pyrrolidone) (PVP) polymer, or a combination thereof.

4. The nanowire array of claim 3, wherein the nonstick layer comprises poly(propylene glycol) diacrylate (PPGDA) polymer.

5. The nanowire array of claim 4, wherein the PPGDA comprises tri (propylene glycol) diacrylate monomers having the following formula:

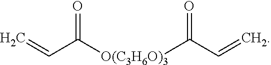

6. The nanowire array of claim 2, wherein:
the semiconductor nanowire comprises GaP;
the inorganic dielectric shell comprises silicon dioxide; and
the nonstick layer comprises a free-standing film or a layer located on a substrate.

7. The nanowire array of claim 1, wherein the nanowires extend substantially orthogonally with respect to a top surface of the nonstick layer and the nonstick layer comprises a free-standing film.

8. The nanowire array of claim 1, wherein the target comprises capture molecules which are configured to bind to a fluorophore labeled analyte biomolecule from an analyte fluid provided to the nanowire array.

9. A biosensor, comprising the nanowire array of claim 1 and radiation source in optical communication with the nanowires.

10. The biosensor claim 9, further comprising a radiation detector in optical communication with the free ends of the nanowires.

11. A sensing method, comprising:
providing a nanowire array nanowires aligned with one another, capture molecules bound to the nanowires, and a nonstick layer surrounding fixed ends of the nanowires, the nonstick layer having a lower binding affinity with the capture molecules than the nanowires;
providing an analyte fluid containing an analyte labeled with a fluorophore to the nanowire array;
selectively binding the analyte labeled with the fluorophore to the capture molecules;
exposing the array to source radiation;
waveguiding emitted radiation from the fluorophores through the nanowires to a radiation detector; and
determining a presence of the analyte in the analyte fluid based on detected emitted radiation.

12. The method claim 11, wherein:
each nanowire comprises a semiconductor nanowire surrounded by an inorganic dielectric shell;
the nonstick layer comprises a polymer layer; and
the analyte comprises a biomolecule.

13. The method claim 12, wherein the nonstick layer comprises a polyacrylate polymer, a polyethylene glycol (PEG) polymer, a poly(ethylene oxide) (PEO) polymer, a polyvinyl alcohol (PVA) polymer, a poly(vinyl pyrrolidone) (PVP) polymer, or a combination thereof.

14. The method of claim 13, wherein the nonstick layer comprises poly(propylene glycol) diacrylate (PPGDA) polymer.

15. A method of forming a nanowire array, comprising:
providing a nanowire array comprising nanowires aligned with one another and a nonstick layer surrounding fixed ends of the nanowires; and
providing target molecules to the nanowire array such that the target molecules selectively bind to the nanowires relative to the nonstick layer.

16. The method of claim 15, wherein the nonstick layer has a lower binding affinity with the target molecules than the nanowires.

17. The method of claim 15, wherein the target comprises capture molecules which are configured to bind to a fluorophore labeled analyte biomolecule from an analyte fluid provided to the nanowire array.

18. The method of claim 17, further comprising providing the nanowire array into biosensor comprising a radiation source in optical communication with free ends of the nanowires, and a radiation detector in optical communication with the free ends of the nanowires.

19. The method of claim 15, further comprising:
growing the nanowires in a vapor phase;
providing the nanowires into a polymer precursor containing monomers;
aligning the nanowires; and
polymerizing the polymer precursor to form the nonstick layer.

20. The method of claim 15, wherein:
each nanowire comprises a semiconductor nanowire surrounded by an inorganic dielectric shell; and
the nonstick layer comprises a polyacrylate polymer, a polyethylene glycol (PEG) polymer, a poly(ethylene oxide) (PEO) polymer, a polyvinyl alcohol (PVA) polymer, a poly(vinyl pyrrolidone) (PVP) polymer, or a combination thereof.
